(19) United States
(12) Patent Application Publication
(10) Pub. No.: US 2010/0305699 A1
(43) Pub. Date: Dec. 2, 2010

(54) STRUCTURE OF NATURAL OR ARTIFICIAL HAIR FOR TRANSPLANTATION AND HAIR TRANSPLANTATION METHOD USING THE SAME

(76) Inventor: Youn Sung Kim, Daejeon (KR)

Correspondence Address: JENKINS, WILSON, TAYLOR & HUNT, P. A., 3100 Tower Blvd., Suite 1200, DURHAM, NC, 27707, US

(21) Appl. No.: 12/809,716

(22) PCT Filed: Oct. 27, 2008
(86) PCT No.: PCT/KR2008/006323

§ 371 Date: Aug. 12, 2010

(30) Foreign Application Priority Data
Dec. 31, 2007 (KR) 10-2007-0141606
Feb. 14, 2008 (KR) 10-2008-0013644

(51) Int. Cl. A61F 2/10; A41G 5/00

(52) U.S. Cl. 623/15.11; 132/201; 132/56

(57) ABSTRACT

The present invention relates to a structure of a natural or artificial hair for a hair transplant and, more particularly, to a processed structure of a natural or artificial hair for transplantation, comprising: a number of fixing cut parts formed lengthwise on the outer circumferential surface line of a lower end part of a natural or artificial hair to be transplanted to a scalp of a person, that is, on the outer circumferential surface line of an insertion part to be inserted inside the scalp, the fixing cut part cut perpendicular to the outer circumferential surface line of the insertion part and formed along the outer circumference surface of the insertion part while having a predetermined angle toward the center of the hair.

### TECHNICAL FIELD

The present invention relates to a structure of a natural or artificial hair for transplantation and, more particularly, to a processed structure of a natural or artificial hair for transplantation, comprising: a number of fixing alt parts formed at a lower end part of a natural or artificial hair to be transplanted to a scalp of a person, that is, the outer circumferential surface line of an insertion part to be inserted inside the scalp, the fixing cut parts cut perpendicular to and formed along the outer circumferential surface line of the insertion part at a predetermined angle toward the center of the hair.

### BACKGROUND ART

In general, a person who experiences hair loss on the head is under a lot of psychological stress, and specially, a young person who experiences hair loss is likely to get social phobia, making it impossible to function in normal social life.

To solve the aforementioned problems, there have been suggested conventional methods of using a wig or toupee and methods of transplanting hair.

In a conventional method of transplanting hair, a part having thick hair from head or other part of the body of a person is cut to remove a graft containing hair, a bald area is slit, and the graft containing hair is transplanted to the bald area.

### DISCLOSURE OF INVENTION

### Technical Problem

In a conventional method of using a wig or toupee, since a wig or toupee comes off carelessly, a user of the wig or toupee cannot take heavy exercise.

Moreover, since a wig or toupee needs to be individually manufactured to be fit on the head of a user, it takes a long time to manufacture the wig or toupee. In addition, since a wig or toupee is a custom-made article, it is expensive.

Moreover, a wig or toupee needs to be frequently cleaned because it gets dirty by sweat generated from the body of a user. During the dirty wig or toupee is cleaned, since the user has to live in the originally bald head situation, without the wig or toupee, (s)he experiences considerable inconveniences.

In the conventional method of transplanting hair, since the graft containing hair is removed from a part of the body and transplanted to another part of the body, it takes a long time to operate hair transplantation to be finished at once, and a person to undergo the hair transplantation needs to be hospitalized until a wound is healed and the transplanted hair is activated.

Moreover, in the conventional method of transplanting hair, since the surgery for hair transplantation is to be finished at once and the person undergoing the hair transplantation is to be hospitalized, the surgery cost is expensive and the surgery time is long. Thus, the person undergoing the hair transplantation spends a lot of time for the hair transplant.

Therefore, the present invention has been made to solve the above problems, and it is an aspect of the present invention to provide a processed structure of a natural or artificial hair for transplantation, comprising: a number of fixing cut parts formed at a lower end part of a natural or artificial hair to be transplanted to a scalp of a person, that is, the outer circumferential surface line of an insertion part to be inserted inside the scalp, the fixing cut parts cut perpendicular to and formed along the outer circumferential surface line of the insertion part at a predetermined angle toward the center of the hair.

### Technical Solution

In accordance with an embodiment of the present invention, there is provided a processed structure of a natural or artificial hair for transplantation, comprising: a number of fixing cut parts 11 formed at a lower end part of a natural or artificial hair 10 to be transplanted to a scalp 20 of a person, that is, the outer circumferential surface line of an insertion part 14 to be inserted inside the scalp 20, the fixing cut parts 11 cut perpendicular to and formed along the outer circumferential surface line of the insertion part 14 at a predetermined angle toward the center of the hair 10.

The fixing cut part 11 of the hair 10 may be formed in any one of a cone shape, an elliptical cone shape and a polygonal cone shape in its side view, along the outer circumferential surface line of the insertion part 14.

A number of the fixing cut parts 11 may be formed so as to be spaced apart from one another at regular intervals or to be continuously in contact with one another, along the outer circumferential surface line of the insertion part 14.

The insertion part 14 with the fixing cut parts 11 to be inserted into the scalp 20 may be formed by cutting the hair 10 to a necessary length, at any position in a whole length direction of the natural or artificial hair.

The end of the insertion part 14 of the hair 10 may be formed in any one of a round shape or a polygonal shape in its front view.

In a modified example of this embodiment, a processed structure of a hair for transplantation comprises: a fixing attachment 12 having a predetermined length formed on the outer circumferential surface line of an insertion part 14 of a natural or artificial hair 10 to be transplanted to a scalp 20 of a person, wherein the fixing attachment 12 is attached to the entire outer circumferential surface line of the insertion part 14 and an upper end of the fixing attachment 12 is formed at a predetermined angle outward the outer circumferential surface line of the insertion part 14, so that the hair 10 is inserted into the scalp 20 using a surgical operation device.

In another modified example of this embodiment, a processed structure of an artificial hair for transplantation comprises: when forming an artificial hair 10, a number of protruding parts 13 formed on the outer circumferential surface line of an insertion part 14 of the artificial hair 10, at a fixed angle outward the outer circumferential surface line of the insertion part 14 of the artificial hair 10.

The hair 10 may be formed in two-layers by additionally connecting a natural or artificial hair 10 either to an upper part of a natural or artificial hair 10 in which the fixing attachment 12 is attached to the outer circumferential surface line of the insertion part 14 or to an upper part of an artificial hair 10 in which the protrusion parts 13 are formed on the outer circumferential surface line of the insertion part 14.

In accordance with another embodiment of the present invention, there is provided a hair transplantation method using a structure of a natural or artificial hair for transplantation, comprising: a hair cutting step S10 of cutting a natural hair 10 of a person who wants transplantation or an artificial hair 10 in a necessary length for operation, at any position of the hair 10; a fixing cut part cutting step S20 of perpendicularly and inwardly cutting an outer circumferential surface of an insertion part 14 of the hair 10 being cut in step S10 along a length direction of the hair 10, to a predetermined depth and at a predetermined angle, by using a process device; a hair inserting step S30 of inserting the hair 10 with the fixing cut part 11 into a scalp 20 by using a surgical operation device; and a scalp fixing step S40 of fixing the fixing cut part 11 to the scalp 20 by pulling upward the hair 10 inserted into the scalp 20 so that the fixing cut part 11 is unfold.

### Advantageous Effects

As described above, in the processed structure of a natural or artificial hair for transplantation according to the present invention, any part of long hair is cut; an end part being cut from the long hair is cut in a number of times along the outer circumference line thereof; and the hair is inserted into the scalp and is pulled upward so that fixing cut parts are fixed to the scalp. Therefore, it is not required to implant a graft containing hair or to wear or remove a wig or toupee. Furthermore, the hair being transplanted once is semi-permanent.

### BRIEF DESCRIPTION OF THE DRAWINGS

These and other aspects and advantages of the present invention will become apparent and more readily appreciated from the following description of the embodiments, taken in conjunction with the accompanying drawings, in which:

FIG. 1 illustrates hair being cut in a structure of a natural or artificial hair for transplantation according to the present invention;

FIG. 2 illustrates a fixing cut part formed at an insertion part of the hair in the structure of a natural or artificial hair for transplantation according to the present invention;

FIG. 3 illustrates a fixing cut part formed at the insertion part of the hair, along the outer circumferential surface line of the insertion part in the structure of a natural or artificial hair for transplantation according to the present invention;

FIG. 4 is a front sectional view illustrating the hair inserted into a scalp using an insertion device in the structure of a natural or artificial hair for transplantation according to the present invention;

FIG. 5 is a front sectional view illustrating the hair pulled upward after inserted into the scalp in the structure of a natural or artificial hair for transplantation according to the present invention;

FIG. 6 is a front sectional view illustrating the hair fixed to the scalp after inserted into the scalp in the structure of a natural or artificial hair for transplantation according to the present invention;

FIG. 7 is a flow chart of a hair transplantation method using the structure of natural/artificial hair for transplantation according to the present invention;

FIG. 8 illustrates a fixing attachment attached to the insertion part in the structure of a natural or artificial hair for transplantation according to the present invention;

FIG. 9 illustrates a protrusion part formed at the insertion part of the artificial hair in the structure of a natural or artificial hair for transplantation according to the present invention; and

FIG. 10 illustrates the hair formed in two layers in the structure of a natural or artificial hair for transplantation according to the present invention.

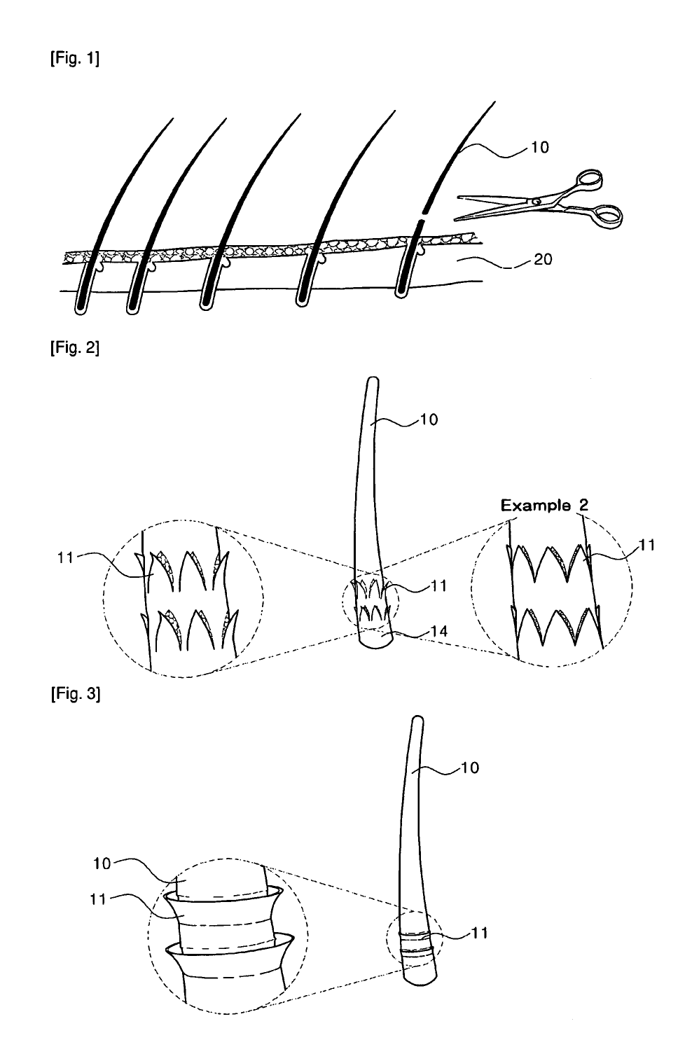

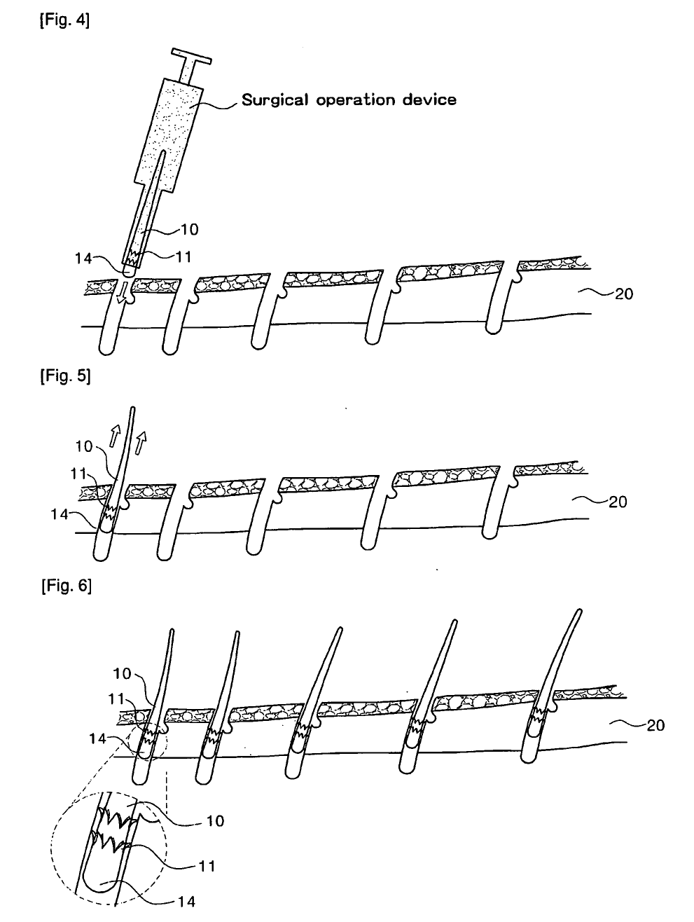

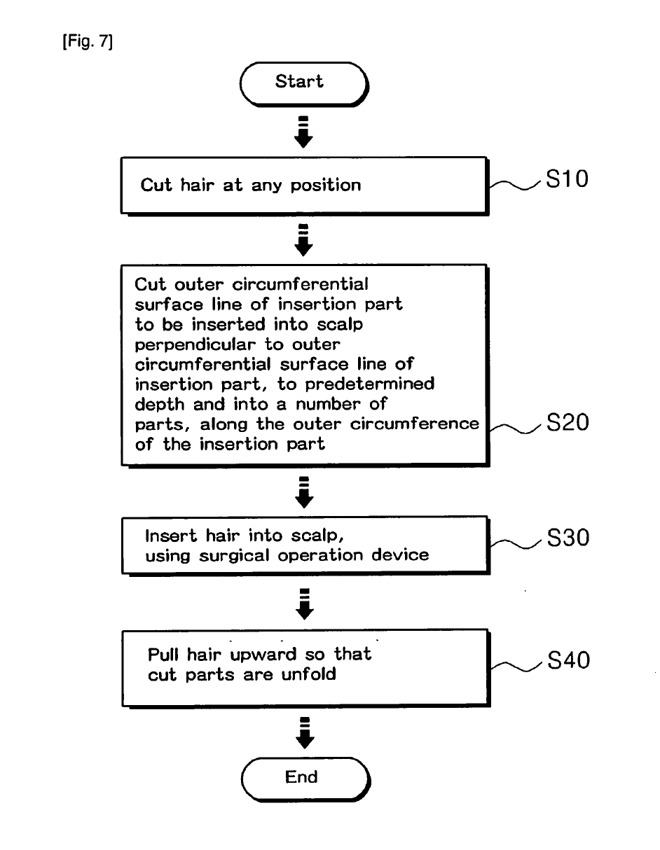

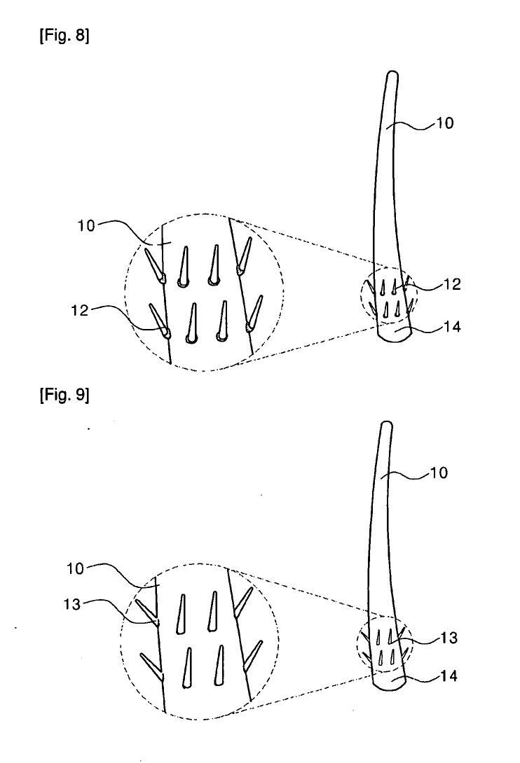

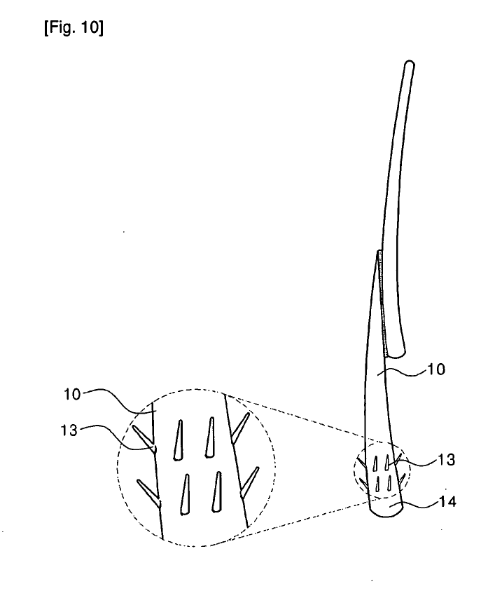

### BRIEF DESCRIPTION OF REFERENCE NUMBERS OF MAJOR ELEMENTS

10: hair 20: scalp

### MODE FOR THE INVENTION

Hereinafter, embodiments of the present invention will be described in detail with reference to the accompanying drawings.

As used herein, the singular forms “a”, “an” and “the” are intended to include the plural forms as well, unless the context clearly indicates otherwise.

A hair 10 of a person to undergo hair transplantation is cut at any length. An end of the hair 10, that is, an insertion part 14 to be inserted into a scalp 20, is perpendicularly cut to its outer circumference line, at a predetermined width, along its outer circumference line (to form a fixing alt part 11), by using a process device. The insertion part 14 is inserted into the scalp 20 by using a surgical operation device and the hair 10 is pulled upward to be fixed to the scalp 20. At this time, the fixing cut part 11 of the insertion part 14 is bent downward so as to have a predetermined angle toward the center of the hair 10. The fixing cut part 11 is formed in any one of a cone shape, an elliptical cone shape and a polygonal cone shape which are pointed upward in its front view.

In a modified example of the embodiment, instead of the fixing cut part 11 formed on the insertion part 14, a fixing attachment 12 may be formed on the outer circumferential surface line of the insertion part 14. In this case, the fixing attachment 12 attached to the outer circumferential surface line of the insertion part 14 has an upper part at a predetermined angle outward the outer circumferential surface line of the insertion part 14. The insertion part 14 with the fixing attachment 12 is inserted into the scalp 20 by the surgical operation device.

In another modified example of the embodiment, when forming an artificial hair 10, an unspecified number of protruding parts 13 may be formed on the outer circumferential surface line of the insertion part 14 of the artificial hair at a fixed angle outward the outer circumferential surface line of the insertion part 14.

A hair 10 may be formed in two-layers, by additionally connecting an upper part of a natural or artificial hair 10 in which the fixing attachment 12 is attached to the outer circumferential surface line of the insertion part 14 with another natural or artificial hair 10.

The hair 10 positioned at a lower part of the hair 10 formed in two-layers may be an artificial hair. The artificial hair 10 positioned at the lower part of the hair 10 formed in two-layers may be manufactured to have the protruding parts 13 formed on the insertion part 14 thereof. Then, the other hair 10 is connected to the upper part of the artificial hair 10 having the protruding parts 13.

In the structure of two-layer hair 10, when the two hairs 10 are connected to each other, the natural or artificial hair to be positioned at a lower layer may have the fixing attachment 12 attached to the insertion part 14. The other natural or artificial hair is connected to the upper part of the natural or artificial hair having the fixing attachment 12, wherein the natural or artificial hair to be positioned at a higher layer has no fixing attachment 12 attached to the outer circumferential surface line of the insertion part 14 thereof.

In another structure of the two-layer hair 10, when the two hairs 10 are connected to each other, an upper part of a natural or artificial hair having the fixing attachment 12 may form at one side being in a “¹-” shape of a predetermined length, and a lower part of another natural or artificial hair to be attached to the upper part of the natural or artificial hair may form a “¹-” shape so as to be fit into each other.

Then, the connected surfaces of the two-layer hair 10 may be cut obliquely to be connected, so that both parts of the two hairs 10 to be connected are symmetrical to each other. A number of grooves may be formed on the connected surfaces to correspond to each other, to be fit into each other and connected to each other.

What is claimed is:

1. A processed structure of a natural or artificial hair for transplantation, comprising:
a number of fixing cut parts (11) formed at a lower end part of a natural or artificial hair (10) to be transplanted to a scalp (20) of a person, that is, the outer circumferential surface line of an insertion part (14) to be inserted inside the scalp (20), the fixing cut parts (11) cut perpendicular to the outer circumferential surface line of the insertion part (14) and formed along the outer circumferential surface line of the insertion part (14) at a predetermined angle toward the center of the hair (10).

2. The processed structure of a natural or artificial hair for transplantation according to claim 1, wherein the fixing cut part (11) of the hair (10) is formed in any one of a cone shape, an elliptical cone shape and a polygonal cone shape in its side view, along the outer circumferential surface line of the insertion part (14).

3. The processed structure of a natural or artificial hair for transplantation according to claim 2, wherein the fixing cut parts (11) are formed so as to be spaced apart from one another, at regular intervals, or to be continuously in contact with one another, along the outer circumferential surface line of the insertion part (14).

4. The processed structure of a natural or artificial hair for transplantation according to claim 1, wherein the insertion part (14) with the fixing cut parts (11) to be inserted into the scalp (20) is formed by cutting the hair (10) to a necessary length, at any position in a whole length direction of the natural or artificial hair.

5. The processed structure of a natural or artificial hair for transplantation according to claim 1, wherein the end of the insertion part (14) of the hair (10) is formed in any one of a round or a polygonal shape in its front view.

6. A processed structure of a natural or artificial hair for transplantation comprising:
a fixing attachment (12) having a predetermined length formed on the outer circumferential surface line of an insertion part (14) of a natural or artificial hair (10) to be transplanted to a scalp (20) of a person, the fixing attachment (12) attached to the entire outer circumferential surface line of the insertion part (14) having an upper end at a predetermined angle outward the outer circumferential surface line of the insertion part (14), so that the hair (10) is inserted into the scalp (20) using a surgical operation device.

7. A processed structure of a natural or artificial hair for transplantation, comprising: when forming an artificial hair (10), a number of protruding parts (13) formed on the outer circumferential surface line of an insertion part (14) of the artificial hair (10), at a predetermined angle outward the outer circumferential surface line of the insertion part (14) of the artificial hair (10).

8. The processed structure of a natural or artificial hair for transplantation according to claim 6, wherein the hair (10) is formed in two-layers, by additionally connecting a natural or artificial hair (10) either to an upper part of a natural or artificial hair (10) in which the fixing attachment (12) is attached to the outer circumferential surface line of the insertion part (14), or to an upper part of an artificial hair (10) in which the protruding parts (13) are formed on the outer circumferential surface line of the insertion part (14).

9. A hair transplantation method using a structure of a natural or artificial hair for transplantation, comprising:
a hair cutting step (S10) of cutting a natural hair (10) of a person who wants transplantation or an artificial hair (10) in a necessary length for operation, at any position of the hair (10);
a fixing cut part cutting step (S20) of perpendicularly and inwardly cutting an outer circumferential surface of an insertion part (14) of the hair (10) being cut in step (S10) along a length direction of the hair (10), to a predetermined depth and at a predetermined angle, by using a process device;
a hair inserting step (S30) of inserting the hair (10) with the fixing cut part (11) into a scalp (20) by using a surgical operation device; and
a scalp fixing step (S40) of fixing the fixing cut part (11) to the scalp (20) by pulling upward the hair (10) inserted into the scalp (20) so that the fixing cut part (11) is unfold.
